(19) United States
(12) Patent Application Publication
(10) Pub. No.: US 2003/0190692 A1
(43) Pub. Date: Oct. 9, 2003

(54) Method for measuring the activity of the blood clotting factor XIIIA

(76) Inventors: Dagmar Prasa, Erfurt (DE); Jorg Sturzebecher, Erfurt (DE)

Correspondence Address: JORDAN AND HAMBURG LLP, 122 EAST 42ND STREET, SUITE 4000, NEW YORK, NY, 10168, US

(21) Appl. No.: 10/380,366

(22) PCT Filed: Sep. 6, 2001
(86) PCT No.: PCT/EP01/10285

(30) Foreign Application Priority Data
Sep. 13, 2000 (DE) 100 46 187.5

(51) Int. Cl. C12Q 1/56

(52) U.S. Cl. 435/13

(57) ABSTRACT

The invention relates to a method for measuring the activity of the blood clotting factor XIIIa. The aim of the invention is to provide a method for measuring the activity of FXIIIa which is simple to carry out and which is also suitable for use with testing systems that have a high throughput. It was found that activated FXIIIa influences the light transmitting power of fibrin clots. Fibrinogen is caused to clot with a fibrinogen-splitting enzyme. The change in the light transmitting power of the samples according to the concentration of FXIIIa present is registered and is used as a measure of the activity of FXIIIa. The inventive method can be used especially for screening for FXIIIa inhibitors.

### BACKGROUND OF THE INVENTION

The present invention relates to a method for measuring the activity of the blood clotting factor XIIIa, in particular for the application of test systems that have a high throughput.

The blood clotting factor XIIIa (FXIIIa) is as transglutaminase the sole enzyme not having a protoelytic effect in the plasmatic coagulation system. The inactive proenzyme factor XIII (FXIII) circulating in the plasma is being activated by thrombin during the blood coagulation process and catalyzes cross-linking of fibrin monomer molecules and incorporation of other plasma proteins (such as α₂-antiplasmin) in the fibrin network by the formation of ε-(-γ-glutamyl)-lysine compounds. Due to this effect, the mechanic strength and resistance of the fibrin clot to fibrinolytic degradation are increased.

Apart from blood coagulation, FXIIIa has a function in wound healing processes. FXIII has also been detected in various cells (thrombocytes, macrophage) and tissues (placenta). To date, the function(s) of cellular FXIIIa is (are) less known (L. Muszbek et al.: Thromb. Res. 94, 1999, 271-305).

However, active, specific inhibitors are not available to further clarify the physiological role of FXIIIa. The therapeutic application of such inhibitors for thrombotic prophylaxis purposes is in discussion, the combination with plasminogen activators supports the lysis of thrombotic closures. (E. M. Leidy et al.: Thromb. Res. 59, 1990, 15-26; R. J. Shebuski et al.: Blood 75, 2000, 1455-1459).

Till now, only a small number of FXIIIa inhibitors have been described. Mostly, these are substrates belonging to the type of substituted alkylamin which are linked with the glutamine residues in fibrin thus preventing fibrin cross-linking. However, to produce a sufficiently strong effect, high concentrations of these pseudosubstrates are required (S.-Y. Kim et al.: Biochem. Biophys. Res. Commun. 233, 1997, 39-44).

Among others, imidazole derivatives, triazole and tetrazole compounds have been described as direct inhibitors of FXIIIa. They block the sulfhydryl group in the active center of the enzyme irreversibly (U.S. Pat. No. 5,077,285; U.S. Pat. No. 5,177,092; U.S. Pat. No. 5,047,416).

The FXIIIa inhibitor L722151, a thiazolo thiadiazolium derivative, developed by the Merck Sharp & Dohme company, is able to improve the thrombolysis by a plasminogen activator in animal models (E. M. Leidy et al.: Thromb. Res. 59, 1990, 15-26).

Apart from synthetic inhibitors, natural inhibitors of FXIIIa are also known. The high-performance tridegin has been isolated from the haementeria ghilianii leech, whereas other inhibitors have been gained from other microorganisms (U.S. Pat. No. 6,025,330; U.S. Pat. No. 5,710,174; K. Ikura et al.: Biosci. Biotechnol. Biochem. 64, 2000, 116-124).

The common methods for measuring FXIIIa activity are based on the following two principles:

(1) The clot solubility test uses the different solubility of crosslinked and non crosslinked fibrinogen clots in urea. However, it only permits a half-quantitative analysis of FXIIIa activity (P. Sigg: Thromb. Diath. Haemorrh. 15, 1966, 238-251; A. A. Tymiak et al.: J. Antibiotics 46, 1993, 204-206).

(2) The catalytic feature of FXIIIa is utilized to bind marked or biotinylized amine substrates (dansylcadaverin, biotinpentylamine) to normally immobilized peptides (such as casein or fibrin). The determination of the bound substrate is carried out directly (fluorescense or chemiluminescense modifications) or via antibody- or streptavidine-coupled enzymes (U.S. Pat. No. 5,015,588; U.S. Pat. No. 4,601,977). All these methods require several incubation steps and are therefore time-consuming and labor-intensive and not suited for the search for new, active and specific inhibitors of FXIIIa, because they do not permit to use high-throughput automated test systems. Likewise, a method in which glycinethylester is linked with a glutamine-containing peptid substrate and the then released ammoniac is measured in an enzymatic reaction is not suitable for high-throughput screening (K. Fickenscher et al.: Thromb. Haemost. 65, 1991, 535-540).

### SUMMARY OF THE INVENTION

The task of the invention is to provide a method for measuring the FXIIIa activity which is simple to carry out and which is suited for screening for FXIIIa inhibitors by applying high-throughput test systems in particular.

It was surprising to find that according to its concentration FXIIIa changes the light transmitting power of fibrin clots—generated by the effect of a fibrinogen-splitting enzyme, such as thrombin or batroxobin. On the basis of these results, a method has been developed for measuring the FXIIIa activity. It is as follows:

Fibrinogen is caused to coagulate by a fibrinogen-splitting enzyme. Preferably, the snake poison enzyme batroxobin is used. It is a thrombin-like enzyme obtained from the poison of bothrops atrox. Unlike thrombin, it splits only fibrinopeptide A from the fibrinogen molecule. The use of thrombin is also possible. The use of purified fibrinogen gained from human beings or other mammals offers special advantages. It is also possible to work with human anti-coagulated plasma (e.g. by citrate or hirudin) or the one of other mammals as fibrinogen source. The invented method is preferably performed in a buffer solution. TRIS-HCl-buffer with a ph value of 7.4 is mostly used. In order to increase the sensitivity of the test method, a substance—usually a polymer—is added to the preparation solution. This substance is able to change the structure of the developing fibrin fibers in such a way that the light transmitting power of the clot will be reduced. Preferably, polyethyleneglycol 6000 is used in a concentration of 0.2% The increase in extinction (turbidity) caused by fibrinogen coagulation is continuously recorded photometrically over a defined period of time. An end point measurement is also possible. The measurement is best performed at a temperature of 37° C. and a wave length of 405 nm. If activated FXIIIa is added to the preparation solution, the increase in extinction is lower during the reaction, that means that the light transmitting power of the produced clot is higher. The difference between the change in extinction or the area below the extinction-time-curve of a preparation free of FXIIIa and a preparation containing FXIIIa functions as the value for the FXIIIa activity. By means of the test approach just described inhibitors of FXIIIa can be found, because an inhibitor reduces the light transmitting power of the clot according to the intensity of the inhibition effect. The activity of factor XIIIa with and without a test substance is then compared. The difference of the activity is the value for the inhibition effect of the test substance examined. If thrombin is used as the fibrinogen-splitting enzyme, thrombin inhibitors can be searched for in parallel. If the substances examined show an effect of thrombin inhibition, if the fibrinogen coagulation is hindered, the extinction of the sample will not change.

The invented method can be used for measuring the activity of both plasmatic and cellular FXIIIa. The proenzyme FXIII is activated in a separate preparation before starting the measuring process. The activation is performed by thrombin in the presence of Ca ions. In order to prevent the disturbing effect of thrombin on the measuring process, if other fibrinogen-splitting enzymes such as batroxobin are used, a natural or synthetic inhibitor of thrombin is added to the activating preparation solution in a sufficiently high concentration when the complete activation of FXIII has been finished. Preferably, recombinant hirudin is used. If thrombin is used as the fibrinogen-splitting enzyme, this step will not be required.

### DETAILED DESCRIPTION OF THE INVENTION

The present invention is further illustrated with reference to the following four examples demonstrated in the figures.

The figures show:

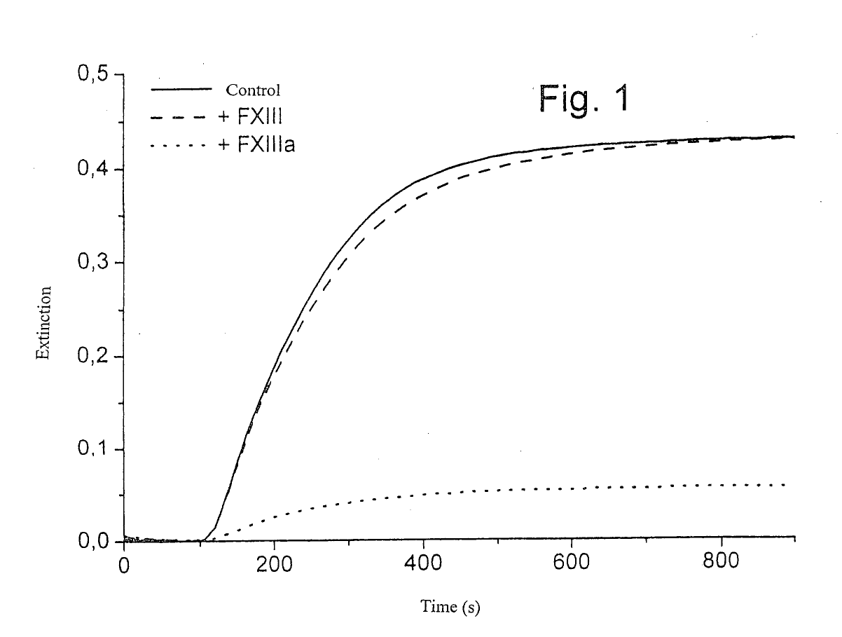

FIG. 1: Coagulation of citrate-anticoagulated plasma with batroxobin in the presence of activated FXIII

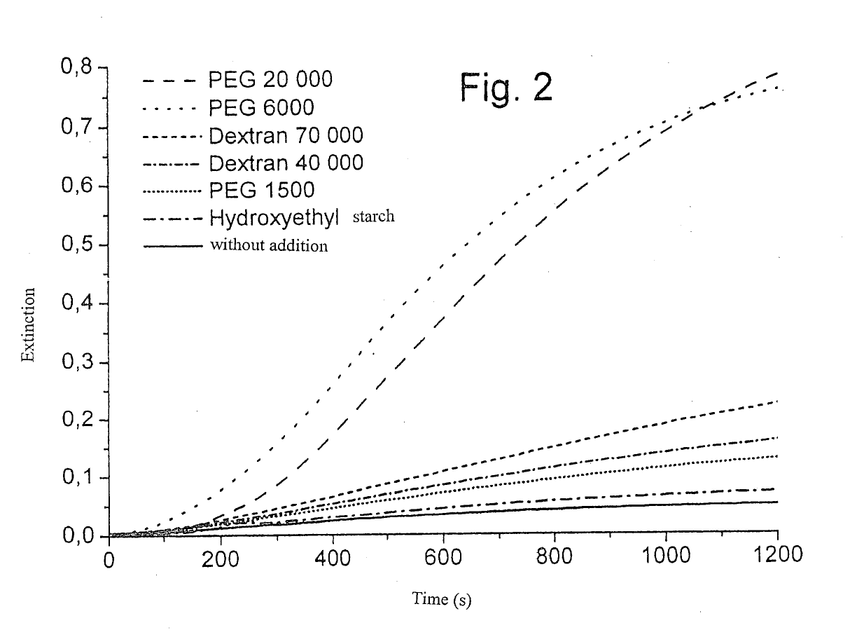

FIG. 2: Influence of various polymers on the coagulation of fibrinogen with batroxobin

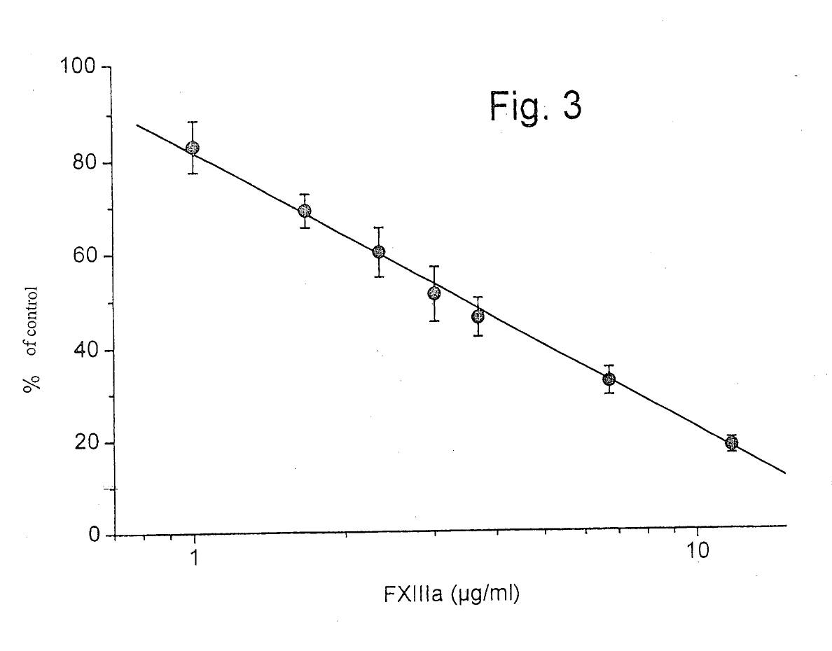

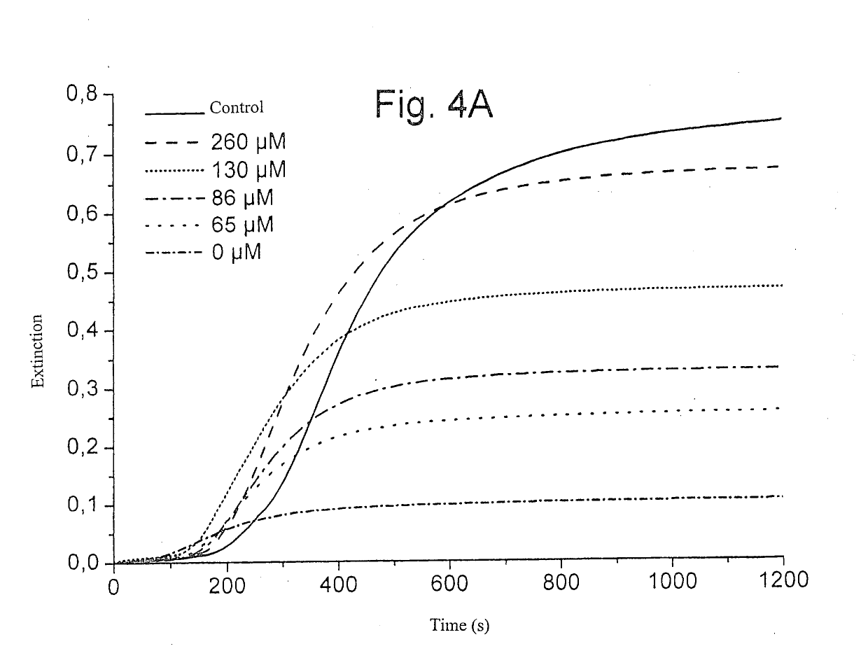

FIG. 3: Fibrinogen coagulation by batroxobin in the presence of different FXIIIa concentrations

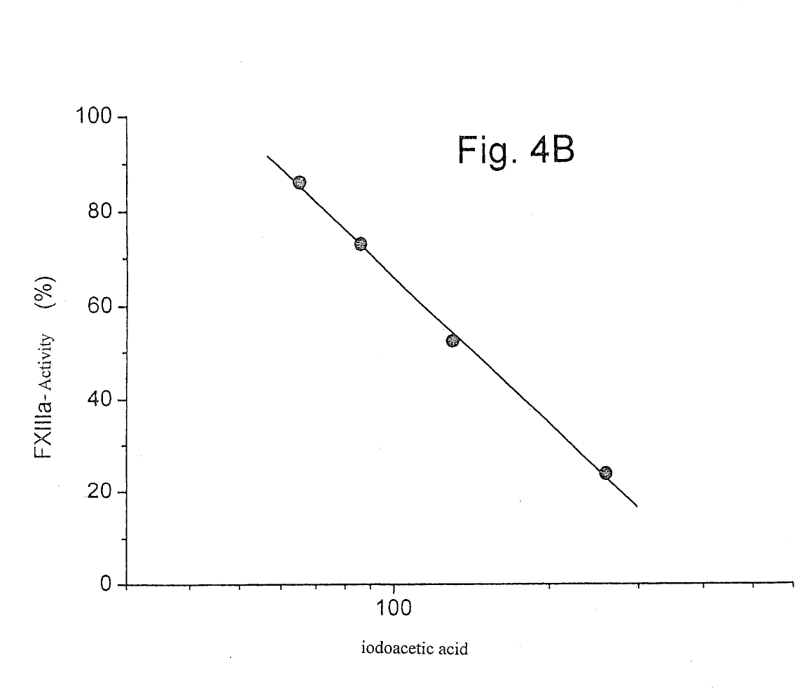

FIG. 4: Inhibition of FXIIIa by means of iodoacetic acid

FIG. 4A: Measurement curve

FIG. 4B: FXIIIa activity as a function of inhibitor concentration

### EXAMPLE 1

Coagulation of Citrate-Anticoagulated Plasma with Batroxobin in the Presence of Activated FXIII

Activation of FXIII:

865 μl of TRIS buffer (0.05 M; 0.154 M NaCl; pH 7.4), 25 μl of CaCl₂ (0.2 M), 100 μl of thrombin (from beef plasma, 100 U/ml) and 10 μl of cellular recombinant FXIII (Aventis-Behring, Marburg, Germany; 8.6 mg/ml or 1155 U/ml) are mixed and incubated at 37° C. for 10 min. Afterwards, 25 μl of recombinant hirudin (HBW 023, Höchst, Frankfurt, Germany; 1000 E/ml) are added.

Preparation Solution:

100 μl of citrate plasma, 60 μl of TRIS buffer (pH 7.4) and 40 μl of the activation solution described above are mixed on a microtiter plate and tempered to 37° C. The reaction is started by adding 50 μl of batroxobin (Pentapharm Ltd., Basel, Switzerland; 2.5 U/ml). The increase in extinction is measured over 15 min at a temperature of 37° C. and at a wave length of 405 nm in a microtiter plate photometer (iEMS, Labsystems, Helsinki, Finland). A preparation solution free of factor XIIIa is used for checking purposes.

The measurement curves are shown in FIG. 1.

Result:

After a short lag period, the extinction increases with the start of the coagulation process and approaches a plateau value in the last part of the measurement curve. In the presence of activated FXIII the increase in extinction is considerably lower than in samples free of FXIIIa. Non-activated FXIII does not influence the light transmitting power of the clot.

### EXAMPLE 2

Influence of Various Polymers on the Coagulation of Fibrinogen with Batroxobin

100 μl of fibrinogen (Sigma, Deisenhofen, Germany; 0.6%); 25 μl of a polymer (25 mg/ml); 75 μl of TRIS buffer are tempered onto 37° C. on a microtiter plate. After the addition of 50 μl of batroxobin (2.5 U/ml) the increase in extinction is measured over a period of 20 min under the conditions described in example 1.

The influence of hydroxyethyl starch, dextran sulfate and polyethyleneglycol on the light transmitting power of fibrinogen clots is demonstrated in FIG. 2.

Result:

The added polymers cause a reduced light transmitting power of the clots, an effect which becomes obvious in the higher increase in extinction. The influence of hydroxyethyl starch, dextran and polyethyleneglycol 1500 is relatively low. Polyethyleneglycol 6000 and 20000, however, increase the extinction by a factor of about 15 compared with samples free of polymers.

### EXAMPLE 3

Fibrinogen Coagulation by Batroxobin in the Presence of Different FXIIIa Concentrations

The activation of FXIII is performed according to example 1.

On a microtiter plate, 100 μl of fibrinogen (0.6%), 25 μl of PEG 6000 (2%), 100 μl of activation solution (with different FXIIIa concentrations) are mixed and after being tempered onto 37° C. charged with 25 μl of batroxobin (5 E/ml). The extinction is measured according to the description given in example 2. The extinction increase of the samples is given in % in relation to the extinction increase of a sample free of FXIIIa (control=100%).

FIG. 3 shows the calibration curve for FXIIIa. The figure depicts the mean values (n=6)±standard deviation.

Result:

Depending on its concentration, FXIIIa increases the light transmitting power of the clots. The calibration curve is linear, if FXIIIa concentration values vary between 1 and 12 μg/ml.

### EXAMPLE 4

Inhibition of FXIIIa by Iodoacetic Acid

The activation of FXIII is performed as described in example 1.

100 μl of fibrinogen (0.6%), 25 μl of PEG 6000 (2%), 80 μl of iodoacetic acid in buffer (0.2-0.8 mM) and 20 μl of the activation solution are mixed and tempered onto 37° C. When 25 μl of batroxobin (5 U/ml) have been added, the extinction is measured according to the explanation given above.

The measurement curves are shown in FIG. 4A. FIG. 4B illustrates the FXIIIa activity as a function of the inhibitor concentration.

Result:

Iodoacetic acid inhibits the activity of FXIIIa according to its concentration. This effect becomes obvious in the reduced light transmitting power of the clot. The inhibitor concentration (IC₅₀) which causes a reduction of the FXIII activity by 50% is determined as a measure for the inhibiting effect. The IC₅₀ value for iodoacetic acid is 147 μM.

1. A method for measuring the activity of the clotting factor XIIIa in a sample solution which contains FXIIIa or in which factor XIIIa is formed, wherein fibrinogen is added to said sample solution and caused to coagulate by a fibrinogen-splitting enzyme, and the resulting change in extinction is measured and evaluated.

2. The method according to claim 1, wherein batroxobin is used as the fibrinogen-splitting enzyme.

3. The method according to claim 1, wherein thrombin is used as the fibrinogen-splitting enzyme.

4. The method according to claims 1 and 2, wherein anticoagulated plasma is used as the source of fibrinogen.

5. The method according to claims 1 through 3, wherein a substance, a polymer in particular, is added to the preparation solution used for measuring the extinction and which reduces the light transmitting power of the clot.

6. The method according to claim 5, wherein polyethyleneglycol 6000 is used as the polymer.

7. The application of the present method according to claims 1 through 6 for screening inhibitors of FXIIIa.

8. The application of the present method according to claim 3 for the simultaneous screening both of inhibitors of FXIIIa and thrombin.
